(19) United States
(12) Patent Application Publication
(10) Pub. No.: US 2012/0122081 A1
(43) Pub. Date: May 17, 2012

(54) DIFFERENTIATING PICORNA VIRUSES, NUCLEIC ACIDS THEREFOR, USE THEREOF AND BIOASSAY METHODS EMPLOYING THEM

(76) Inventors: Matti Waris, Turku (FI); Riikka Österback, Turku (FI); Timo Hyypiä, Turku (FI)

(21) Appl. No.: 13/322,904

(22) PCT Filed: May 28, 2010
(86) PCT No.: PCT/FI10/50429

§ 371 Date: Jan. 30, 2012

(30) Foreign Application Priority Data
May 28, 2009 (FI) 20095592

(51) Int. Cl. C12Q 1/70; C07H 21/02; C07H 21/04

(52) U.S. Cl. 435/5; 536/24.32

(57) ABSTRACT

A nucleic acid comprising a 13 base sequence selected from the group consisting of tcGg TtccgCt Gc, tcGgTtccgCc Ac, tcGgTcCcaTcCc, tcGgTtCcaTcCc, ttGgTcCcaTcCc, ttGgTtCcaTcCc, tcGgTcccgTcCc, and tcGgTtccgTcCc, where A and a are adenine, C and c are cytosine, G and g are guanine, and T and t are thymine or uracil, and complementary sequences thereof; provided that only a very limited number of additional bases are included at the 5′ and 3′ ends of the sequences. The use of these nucleic acids for differentiating picornaviruses, and bioassay methods employing these nucleic acids in nucleic acid amplification assays, are also disclosed.

### FIELD OF THE INVENTION

This invention relates to differentiation of pikornaviruses, nucleic acids therefore, use thereof and bioassays employing them. More specifically this invention relates to differentiating between enteroviruses and rhinoviruses and/or determining enteroviruses and/or rhinoviruses.

### BACKGROUND OF THE INVENTION

The publications and other materials used herein to illuminate the background of the invention, and in particular, cases to provide additional details respecting the practice, are incorporated by reference.

US 2005/02039055 discloses a diagnostic method for differentiating enteroviruses and probes for use in said method. US 2005/0239055 does not relate to rhinoviruses.

Lönnrot et al., Journal of Medical Virology 59:378-384 (1999); Kares et al., Journal of Clinical Virology 29 (2004) 99-104 and Jokela et al., Journal of Clinical Microbiology, March 2005, p. 1239-1245 disclose probes for determining enteroviruses or rhinoviruses.

Sequences disclosed in KR 20080111785, US 2002/160976, US 2003/104410, US 2006/051769, US 2007/009899, US 2007/092871, US 2007/141575, US 2007/243546, U.S. Pat. No. 6,258,570, WO 01/47944, WO 2004/044123 WO 2004/048511, WO 2007/150071 and Kiang et al., Clin. Microbiol. March 2008, Vol. 46, No. 11. pp. 3736-3745 relate to sequences of the present invention.

### OBJECT AND SUMMARY OF THE INVENTION

One object of the present invention is to provide nucleic acid sequences useful for differentiating picornaviruses.

Another object of the present invention is to provide use of nucleic acid sequences for differentiating picornaviruses between enteroviruses and rhinoviruses.

A further object of the present invention is to provide a bioassay for differentiating picornaviruses between enteroviruses and rhinovirus.

The present invention provides a nucleic acid comprising a 13 base sequence selected from the group consisting of

| | | |
|---|---|---|
| a) | tcGgTtccgCtGc, | (SEQ ID NO: 1) |
| b) | tcGgTtccgCcAc, | (SEQ ID NO: 2) |
| c) | tcGgTcCcaTcCc, | (SEQ ID NO: 3) |
| d) | tcGgTtCcaTcCc, | (SEQ ID NO: 4) |
| e) | ttGgTcCcaTcCc, | (SEQ ID NO: 5) |
| f) | ttGgTtCcaTcCc, | (SEQ ID NO: 6) |
| g) | tcGgTcccgTcCc, | (SEQ ID NO: 7) |
| h) | tcGgTtccgTcCc, | (SEQ ID NO: 8) |

wherein A and a are adenine, C and c are cytosine, G and g are guanine, and T and t are thymine or uracil, and provided that in any case the nucleic acid does not comprise more than 10 additional bases at either the 5′ end or the 3′ end of the nucleic acid; and
i) if the nucleic acid comprises alternative a) said nucleic acid does not comprise any additional bases at the 5′ end; or, preferably and, does not comprise more than 4, preferably not more than 3, more preferably not more than 2, even more preferably not more than 1 and most preferable not any additional bases at the 3′ end;
ii) if the nucleic acid comprises alternative b) said nucleic acid does not comprise more than 3, preferably not more than 2, more preferably not more than 1 and most preferable not any additional bases at the 5′ end; or, preferably and, does not comprise more than 5, preferably not more than 3, more preferably not more than 2, even more preferably not more than 1 and most preferable not any additional bases at the 3′ end;
iii) if the nucleic acid comprises alternative c) said nucleic acid does not comprise (1) any, (2) more than 2, or (3) more than 4 additional bases at the 5′ end, and does not comprise (1) more than 6, (2) more than 4, or (2) more than 2, respectively, additional bases at the 3′ end, and preferably does not comprise any additional bases at either said 5′end or said 3′ end;
iv) if the nucleic acid comprises alternative d) said nucleic acid does not comprise more than 2, more preferably not more than 1 and most preferable not any additional bases at the 5′ end; or, preferably and, does not comprise more than 2, preferably not more than 1 and most preferable not any additional bases at the 3′ end;
v) if the nucleic acid comprises alternative e) said nucleic acid does not comprise any additional bases at the 5′ end; or, preferably and, not any additional bases at the 3′ end;
vi) if the nucleic acid comprises alternative f) said nucleic acid does not comprise more than 3, preferably not more than 2, more preferably not more than 1 and most preferable not any additional bases at the 5′ end; or, preferably and, does not comprise more than 10, preferably not more than 3, more preferably not more than 2, even more preferably not more than 1 and most preferable not any additional bases at the 3′ end;
vii) if the nucleic acid comprises alternative g) said nucleic acid does not comprise more than 10, preferably not more than 3, more preferably not more than 2, even more preferably not more than 1 and most preferable not any additional bases at the 5′ end; and does not comprise any additional bases at the 3′ end; and
viii) if the nucleic acid comprises alternative h) said nucleic acid does not comprise more than 10, preferably not more than 3, more preferably not more than 2, even more preferably not more than 1 and most preferable not any additional bases at the 5′ end; or, preferably and, does not comprise any additional bases at the 3′ end; and
   complementary sequences thereof.

The present invention also provides a use of nucleic acids comprising a 13 base sequence selected from the group consisting of

| | | |
|---|---|---|
| a) | tcGgTtccgCtGc, | (SEQ ID NO: 1) |
| b) | tcGgTtccgCcAc, | (SEQ ID NO: 2) |
| c) | tcGgTcCcaTcCc, | (SEQ ID NO: 3) |
| d) | tcGgTtCcaTcCc, | (SEQ ID NO: 4) |
| e) | ttGgTcCcaTcCc, | (SEQ ID NO: 5) |
| f) | ttGgTtCcaTcCc, | (SEQ ID NO: 6) |
| g) | tcGgTcccgTcCc, | (SEQ ID NO: 7) |
| h) | tcGgTtccgTcCc, | (SEQ ID NO: 8) |

wherein A and a are adenine, C and c are cytosine, G and g are guanine, and T and t are thymine or uracil; and provided that in any case the nucleic acid does not comprise more than 30, preferably not more than 10, more preferably not more than 3, even more preferably not more than 1 and most preferable not any additional bases at the 5′ end; and does not comprise more than 30, preferably not more than 10, more preferably not more than 3, even more preferably not more than 1 and most preferable not any additional bases at the 3′ end; and
   complementary sequences thereof;
   for differentiating picornaviruses between enteroviruses and rhinoviruses. Characteristic for the use is that at least 2, preferably at least 4, more preferably at least 6 and most preferably 8 different said nucleic acids, each corresponding to one of said nucleic acids with one of sequences a) to h) or said complementary sequence thereof provided that only one of each pair of said sequence and its complementary sequence is used.

The present invention further provides a bioassay method wherein picornaviruses are differentiated between enteroviruses and rhinovirus. The assay is characteristic in that the nucleic acids of the invention defined above are employed in a nucleic acid amplification assay.

### BRIEF DESCRIPTION OF THE DRAWINGS

FIGS. 1A to 1D illustrate the principle of a RT-PCR assay according to the present invention.

FIGS. 2A and 2B illustrate standard curves of threshold values versus log of copies per reaction of bioassays according to the present invention.

### DETAILED DESCRIPTION OF THE INVENTION

The inventors have surprisingly been able to find a rather limited stretch of genome of the picornavirus strains relating to human pathologies (about 200 different strains altogether) which rather limited stretch of genome enables differentiation between rhino- and enteroviruses using probes with short sequences. Moreover, as has been demonstrated in the examples of this application, only a very limited set of probes is sufficient for determining most, if not all, picornavirus strains relating to human pathologies caused by either enteroviruses or rhinoviruses.

Due to the use of probes with short oligonucleotides it is preferred that known means for raising the melting point of the probes are employed. Accordingly many preferred embodiments referred to below relate to such means.

In case some rare picornavirus, which could not be recognized by the enterovirus and rhinovirus probes used, even such a picornavirus could be detected if a general dsDNA label was used and by determining the melting point even such a picornavirus strain could with about 80% certainty be differentiated to be either a enterovirus or rhinovirus based on the melting point.

Differentiation between enterovirus and rhinoviruses is of value due to that possible complications related to enterovirus and rhinoviruses are very different. Complications related to enteroviruses include meningitis, paralysis and myocarditis. Complications related to rhinoviruses include otitis, dyspnea and pneumonia.

### DEFINITIONS

In this disclosure, the term nucleic acid shall be understood to include both natural nucleic acids, such as deoxyribonucleic acid (DNA) and ribonucleic acid (RNA) as well as artificial nucleic acids, such as peptide nucleic acid (PNA), morpholino and locked nucleic acid (LNA), glycol nucleic acid (GNA) and threose nucleic acid (TNA).

The term nucleotide shall in analogy with the above be understood to refer to any nucleoside and one phosphate group, including naturally occurring nucleosides and phosphate groups as well as those included in the artificial nucleic acids as referred to above.

The term modified nucleotide refers to all known modified nucleotides known in the art both naturally occurring and artificial such as those included in the artificial nucleic acids referred to above.

The term locked nucleotide refers to modified nucleotides included in locked nucleic acid (LNA). The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide. The locked ribose conformation enhances base stacking and backbone pre-organization. This significantly increases the thermal stability (melting temperature) of oligonucleotides.

The term complementary sequence shall be understood to refer to that the sequences of each of two strands is complementary to the other in that base pairs between them are non-covalently connected via two or three hydrogen bonds when in hybridized (double-stranded) form. If the sequence of one strand, often referred to as “sense” strand, is known, one can reconstruct a complementary strand, often referred to as “antisense” strand, for such known strand.

Picornaviruses refer to a virus belonging to the family Picornaviridae. Picornaviruses are non-enveloped, positive-stranded RNA viruses with an icosahedral capsid. They are characterized by a single positive-strand genomic RNA known to have a high mutation rate caused by low-fidelity replication and frequent recombination.

Enteroviruses refer to members of the picornavirus family comprising the following species: poliovirus, human enterovirus A (HEV-A) (coxackie A viruses and enterovirus 71), HEV-B (coxsackie B viruses, echoviruses, coxsackie A9 virus, and enteroviruses 69 and 73), HEV-C (coxsackie A viruses), HEV-D (enteroviruses 68 and 70), and at least three animal enterovirus species (bovine, simian, and porcine enteroviruses). In the context of this application referral is particularly to human enterovirus species.

Rhinoviruses refer to members of the picornavirus family comprising the following species: rhinovirus A (HRV-A), rhinovirus B (HRV-B) and rhinovirus C (HRV-C).

Rhinoviruses are the most common viral infective agents in humans, and a causative agent of the common cold.

In the context of this application the term bioassay method refers to a method for detection and/or quantitation of an analyte employing probes recognizing target nucleic acid sequences.

Nucleic acid amplification assay refers to any assay wherein nucleic acids are amplified. Accordingly the term relates to e.g. a polymer chain reaction (PCR) assay or a nucleic acid sequence based amplification assay (NASBA).

The term double stranded nucleic acid dye refers to any dye, e.g. BOXTO (TATAA, Biocenter), known in the art applicable for dying any double stranded nucleic acid.

In the context of this application the term label refers to any atom or molecule that may be directly or indirectly attached to a nucleic acid to provide detectable and preferably quantifiable signal.

### Preferred Embodiments of the Invention

A typical novel nucleic acid of the invention comprises a 13 base sequence selected from the group consisting of

| | | |
|---|---|---|
| a) | tcGgTtccgCtGc, | (SEQ ID NO: 1) |
| b) | tcGgTtccgCcAc, | (SEQ ID NO: 2) |
| c) | tcGgTcCcaTcCc, | (SEQ ID NO: 3) |
| d) | tcGgTtCcaTcCc, | (SEQ ID NO: 4) |
| e) | ttGgTcCcaTcCc, | (SEQ ID NO: 5) |
| f) | ttGgTtCcaTcCc, | (SEQ ID NO: 6) |
| g) | tcGgTcccgTcCc, | (SEQ ID NO: 7) |
| h) | tcGgTtccgTcCc, | (SEQ ID NO: 8) |

wherein A and a are adenine, C and c are cytosine, G and g are guanine, and T and t are thymine or uracil,

If the nucleic acid comprises alternative a) said nucleic acid does not comprise any additional bases at the 5′ end; or, preferably and, does not comprise more than 4, preferably not more than 3, more preferably not more than 2, even more preferably not more than 1 and most preferable not any additional bases at the 3′ end.

If the nucleic acid comprises alternative b) said nucleic acid does not comprise more than 3, preferably not more than 2, more preferably not more than 1 and most preferable not any additional bases at the 5′ end; or, preferably and, does not comprise more than 5, preferably not more than 3, more preferably not more than 2, even more preferably not more than 1 and most preferable not any additional bases at the 3′ end.

If the nucleic acid comprises alternative c) said nucleic acid does not comprise (1) any, (2) more than 2, or (2) more than 4 additional bases at the 5′ end, and does not comprise (1) more than 6, (2) more than 4, or (3) more than 2, respectively, additional bases at the 3′ end, and preferably does not comprise any additional bases at either said 5′end or said 3′ end.

If the nucleic acid comprises alternative d) said nucleic acid does not comprise more than 2, more preferably not more than 1 and most preferable not any additional bases at the 5′ end; or, preferably and, does not comprise more than 2, preferably not more than 1 and most preferable not any additional bases at the 3′ end;

If the nucleic acid comprises alternative e) said nucleic acid does not comprise any additional bases at the 5′ end; or, preferably and, not any additional bases at the 3′ end.

If the nucleic acid comprises alternative f) said nucleic acid does not comprise more than 3, preferably not more than 2, more preferably not more than 1 and most preferable not any additional bases at the 5′ end; or, preferably and, does not comprise more than 10, preferably not more than 3, more preferably not more than 2, even more preferably not more than 1 and most preferable not any additional bases at the 3′ end.

If the nucleic acid comprises alternative g) said nucleic acid does not comprise more than 10, preferably not more than 3, more preferably not more than 2, even more preferably not more than 1 and most preferable not any additional bases at the 5′ end; and does not comprise any additional bases at the 3′ end; and

If the nucleic acid comprises alternative h) said nucleic acid does not comprise more than 10, preferably not more than 3, more preferably not more than 2, even more preferably not more than 1 and most preferable not any additional bases at the 5′ end; or, preferably and, does not comprise any additional bases at the 3′ end.

Typical novel nucleic acids of the invention also include complementary sequences of the sequences defined above.

Typical embodiments of nucleic acids of the invention comprise modified nucleotides, preferably locked nucleotides. In preferred embodiments of these typical embodiments the nucleic acid comprises locked nucleotides, preferably at positions designated above with the capital letters A, C, G, T denoting the base as defined above of said locked nucleotides. In even more preferred embodiments the nucleic acid of the invention comprises 2 to 7, preferably 3 to 6, most preferably 4 or 5 locked nucleotides. Preferably at least nucleotides 10 and 12 of sequences a) to h) and at least nucleotides 2 and 4 of sequences complementary to said sequences a) to h) are locked.

Preferred nucleic acids of the invention are selected from the group consisting of DNA, RNA, LNA, and PNA; preferably LNA and PNA, and most preferably LNA.

In some preferred embodiments of the invention minor groove binder (MGB) technology (Nanogen Inc., San Diego, Calif., USA) can be employed instead or in addition to the use of artificial nucleic acids to increase the thermal stability of the probes.

A typical use of nucleic acids according to the invention involves the use of nucleic acids comprising a 13 base sequence selected from the group consisting of

| | | |
|---|---|---|
| a) | tcGgTtccgCtGc, | (SEQ ID NO: 1) |
| b) | tcGgTtccgCcAc, | (SEQ ID NO: 2) |
| c) | tcGgTcCcaTcCc, | (SEQ ID NO: 3) |
| d) | tcGgTtCcaTcCc, | (SEQ ID NO: 4) |
| e) | ttGgTcCcaTcCc, | (SEQ ID NO: 5) |
| f) | ttGgTtCcaTcCc, | (SEQ ID NO: 6) |
| g) | tcGgTcccgTcCc, | (SEQ ID NO: 7) |
| h) | tcGgTtccgTcCc, | (SEQ ID NO: 8) |

wherein A and a are adenine, C and c are cytosine, G and g are guanine, and T and t are thymine or uracil; and provided that in any case the nucleic acid does not comprise more than 30, preferably not more than 10, more preferably not more than 3, even more preferably not more than 1 and most preferable not any additional bases at the 5′ end; and does not comprise more than 30, preferably not more than 10, more preferably not more than 3, even more preferably not more than 1 and most preferable not any additional bases at the 3′ end; and
   complementary sequences thereof;
   for differentiating picornaviruses between enteroviruses and rhinoviruses. Characteristic for the use is that at least 2, preferably at least 4, more preferably at least 6 and most preferably 8 different said nucleic acids, each corresponding to one of said nucleic acids with one of sequences a) to h) or said complementary sequence thereof provided that only one of each pair of said sequence and its complementary sequence is used.

In preferred embodiments of the invention the nucleic acids used are the novel nucleic acids of the invention as defined by above.

In many preferred embodiments the nucleic acids used do not comprise any additional nucleotides to those defined by the 13 base sequences defined by alternatives a) to h) above and the complementary sequences thereof.

In most preferred embodiments one or more of the nucleic acids used comprise modified nucleotides, preferably locked nucleotides. In these embodiments it is typical that one or more of the nucleic acids comprises locked nucleotides, preferably at positions designated above with the capital letters A, C, G, T denoting the base of said locked nucleotides. Moreover it is typical that one or more of the nucleic acids used comprise 2 to 7, preferably 3 to 6, most preferably 4 or 5 locked nucleotides. It is especially preferred that at least nucleotides 10 and 12 of sequences a) to h) and at least nucleotides 2 and 4 of sequences complementary to said sequences a) to h) are locked.

In many preferred embodiments at least one of the nucleic acids used is a DNA, a RNA, a LNA, or a PNA preferably a LNA or a PNA, and most preferably a LNA.

In preferred embodiments a picornavirus, which cannot be recognized by the probes used, is detected employing a general dsDNA label; and, by determining the melting point, differentiated, based on said melting point, to be either an enterovirus or rhinovirus.

In some preferred embodiments of the invention the nucleic acids of the invention are used as a determiner portion in a priming oligonucleotide, e.g. dual priming oligonucleotide (DPO™), for differentiating picornaviruses, preferably between enteroviruses and rhinoviruses. DPO™ is technology developed by Seegene Inc. (Seoul, South Korea; www.seegene.com). DPO™ comprises of two separate priming regions (a first priming region and a second priming region) joined by a polydeoxyinosine linker. The linker forms like a “bubble-like structure” which itself is not involved in priming, but rather delineates the boundary between two parts of primer. The dual specificity oligonucleotide comprises a 5′-end stabilizer, a polydeoxyinosine linker and a 3′-end portion determiner.

In a typical bioassay method for differentiating picornaviruses between enteroviruses and rhinovirus the novel nucleic acids defined above are employed in a nucleic acid amplification assay. Not only the novel nucleic acids of the invention, but also those defined above for use in the invention can be used in bioassays according to the invention. Preferably the nucleic acid amplification assay is a polymer chain reaction (PCR) assay or a nucleic acid sequence based amplification assay (NASBA), preferably a PCR assay.

Labels employed in the present invention may be detectable through signal generated by fluorescence, luminescence, radioactivity, enzymic activity, to name a few. For example, labels such as FAM (6-carboxyfluorescein), Cy5, ROX, Yakima Yellow or other fluorochromes may be utilized. For example, dual label probes can be made by adding a fluorochrome such as FAM or Cy5 to the 5′ end of the oligonucleotide and a quencher such as Black Hole Quencher (BHQ) or Dark Quencher (DQ) to the 3′ end of the oligonucleotide. Such probes have relatively low fluorescence when intact, but when the probe is cleaved by the exonuclease activity of the polymerase in PCR, the fluorescence increases as the fluorochrome becomes separated from the quencher. Other principles of probe technologies, such fluorescence resonance energy transfer (FRET) probes, or Molecular Beacons, may also be applied if appropriate modifications are made to the assay reagents.

In many preferred embodiments the nucleic acid amplification assay is a PCR assay and it comprises the steps of

a) nucleic acid extraction from a sample,
b) reverse transcription,
c) PCR amplification employing nucleic acids defined above, and
d) analysis of amplification results.

In many preferred embodiments of the bioassay method

i) at least one nucleic acid as defined in a) and b) and at least one nucleic acid as defined in c) to g) above are employed, and
ii) said nucleic acid as defined in a) and b) are labelled with a first label detectable at a first wavelength and said nucleic acids as defined in c) to g) are labelled with a second label detectable at a second wavelength.

In especially preferred embodiments of the bioassay method a double stranded nucleic acid dye, preferably BOXTO (TATAA, Biocenter), detecting any nucleic acid amplification, preferably at a third wavelength, is further employed.

In many preferred embodiments of the bioassay method a multichannel real time PCR instrument employing at least two, preferably at least three, different wavelength channels is used in step c).

In many preferred embodiments of the bioassay method is a PCR assay and step d) as defined above comprises melting point determination of the end products of PCR amplification.

In many preferred embodiments of the bioassay method dilution series of samples are employed and results are compared to standards with known copy numbers of genes detected in order to obtain quantitative results.

### EXAMPLES

The following experimental section illustrates the invention by providing examples of differentiations carried out by the method of the invention using probes of the invention.

### Methodology

### I. Sample

Any sample that is suitable for down-stream extraction of nucleic acids in the sample can be used in this method. Typical samples are clinical specimens from patients suspected to be infected with picornaviruses. A clinical specimen can be e.g. a nasal swab, a throat swab, nasopharyngeal aspirate, middle ear fluid, bronchoalveolar lavage, vesicular fluid, stool, cerebrospinal fluid, serum, plasma, or tissue obtained by biopsy or autopsy. Typical samples are also cells and cell culture supernatants from cell cultures infected with picornaviruses or clinical specimens suspected to contain picornaviruses.

### II. Nucleic Acid Extraction.

Any method for nucleic acid extraction or other type of method resulting in RNA of the original sample in such form that the RNA is suitable for the downstream reverse transcription reaction can be applied. RNA extracts were stored at −70° C. In this work, the following methods have been used:

a. E.Z.N.A. Viral RNA Isolation Kit (Omega Bio-Tek) according to manufacturer's protocol.
b. NucliSense easyMag automated nucleic acid extraction (BioMerieux) according to manufacturer's protocol.

### III. Reverse Transcription

The purpose of the reverse-transcription is to obtain complementary DNA (cDNA) whenever a sample contains RNA that includes the sequence for the picornavirus specific ENRI (4-) primer. Another picornavirus specific oligonucleotide, priming the reverse transcription of the sequences determined by the probes, could be used instead of the ENRI (4-) primer. It is also possible to use random primers in order to make cDNA to any RNA present in the sample.

For reverse transciption of RNA to cDNA, an RT mix was prepared, keeping all components and prepared mix ice-cold. Following RT mixes were used:

a.

| | |
|---|---|
| Water | 9.0 μl |
| 5x Buffer (Promega #M530A) | 8.0 μl |
| 10 mM dNTP (Pharmacia #27-2035-01) | 8.0 μl |
| 10 μM ENRI(4-) primer (GAAACACGGACACCCA | 4.8 μl |
| AAGTA; SEQ ID NO: 9) | |
| 40 U/μl Rnase inhibitor (Promega #N2511) | 0.1 μl |
| 200 U/μl M-MLV reverse transcriptase | 0.1 μl |
| (Promega #M530A) | |
| Total volume | 30.0 μl |

b.

| | |
|---|---|
| Water | 4.4 μl |
| 5x Buffer (Fermentas #K1623) | 4.0 μl |
| 10 mM dNTP (Fermentas #K1623) | 4.0 μl |
| 10 μM ENRI(4-) primer (GAAACACGGACACCCAA | 2.4 μl |
| AGTA; SEQ ID NO: 9) | |
| 40 U/μl Rnase inhibitor (Fermentas #K1623) | 0.1 μl |
| 200 U/μl M-MLV reverse transcriptase | 0.1 μl |
| (Fermentas #K1623) | |
| Total volume | 15.0 μl |

Reverse transcription (RT) was performed by adding 10 or 5 μl of sample RNA to 30 or 15 μl of RT mix, respectively, in a 0.5 ml microcentrifuge tube and incubating at 42° C. for 60 min, then at 70° C. for 10 min, and then chilled on ice. If not further processed immediately, resulting cDNAs were stored at −20° C.

### IV. PCR

For the amplification of the cDNA in PCR, the following PCR mix was prepared keeping all reagents ice-cold.

| | |
|---|---|
| Water | 3.25 μl |
| 10 μM BOXTO (TATAA Biocenter) | 1.0 μl |
| 2x QuantiTect Probe PCR Master Mix | 12.5 μl |
| (Qiagen #204343) | |
| 10 μM ENRI(4-) (GAAACACGGACACCCAAAGTA; | 1.5 μl |
| SEQ ID NO: 9) | |
| 10 μM ENRI(3+) (CGGCCCTGAATGCGGCTAA; | 1.5 μl |
| SEQ ID NO: 10) | |
| 5 μM rhinovirus probes A (SEQ ID NOS: | 0.5 μl |
| 3, 4, 5 and 6) | |
| 5 μM rhinovirus probes B (SEQ ID NOS: | 0.25 μl |
| 7 and 8) | |
| 5 μM enterovirus probe 1 (SEQ ID NO: 1) | 0.25 μl |
| 5 μM enterovirus probe 2 (SEQ ID NO: 2) | 0.25 μl |
| Total volume | 20.0 μl |

All rhinovirus probes comprised a green FAM label and a DQ quencher. All enterovirus probes comprised a red Cy5 label and a DQ quencher, Both rhinovirus probe mixtures A and B were 5 μM with respect to each of the probes in the mixture,

A 5 μl aliquot of the cDNA preparation was added into 20 μl of the PCR mix, and subjected to amplification in a real-time PCR instrument (Rotor-Gene, Corbett Research, Australia). Cycling parameters in the amplification were: 15 min at 95° C.; 50 cycles of 15 s at 95° C., 30 s at 65-55° C. with 1° C./cycle touchdown during the first 10 cycles, and 40 s at 72° C.; followed by melting curve generation at 72-95° C., raising by 1° C. every 5 s. Amplifications were monitored on green, yellow, and red channels, and melting curves on the yellow channel of the instrument.

Results of PCR were analysed using software of the instrument and cycle threshold (Ct) values were recorded for each sample on each channel, and melting points on yellow channel. The cycle threshold value is the cycle number, when the fluorescence in the sample tube crosses the threshold set for positive amplifications. Thus, a strongly positive sample gives a lower Ct value than a weakly positive sample.

### Results

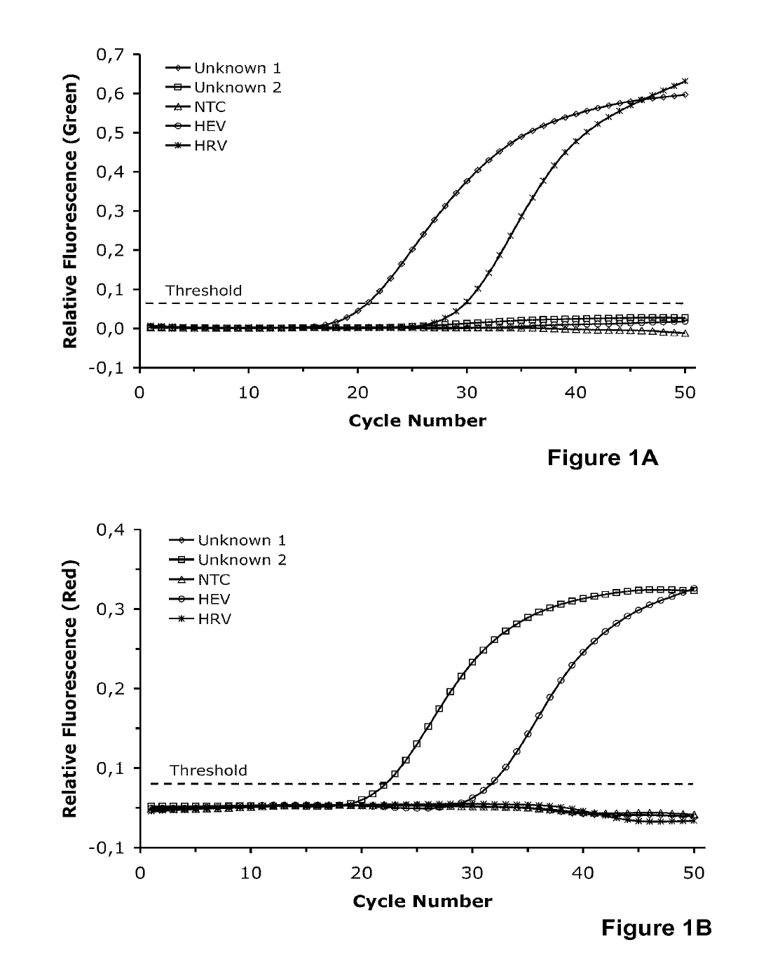

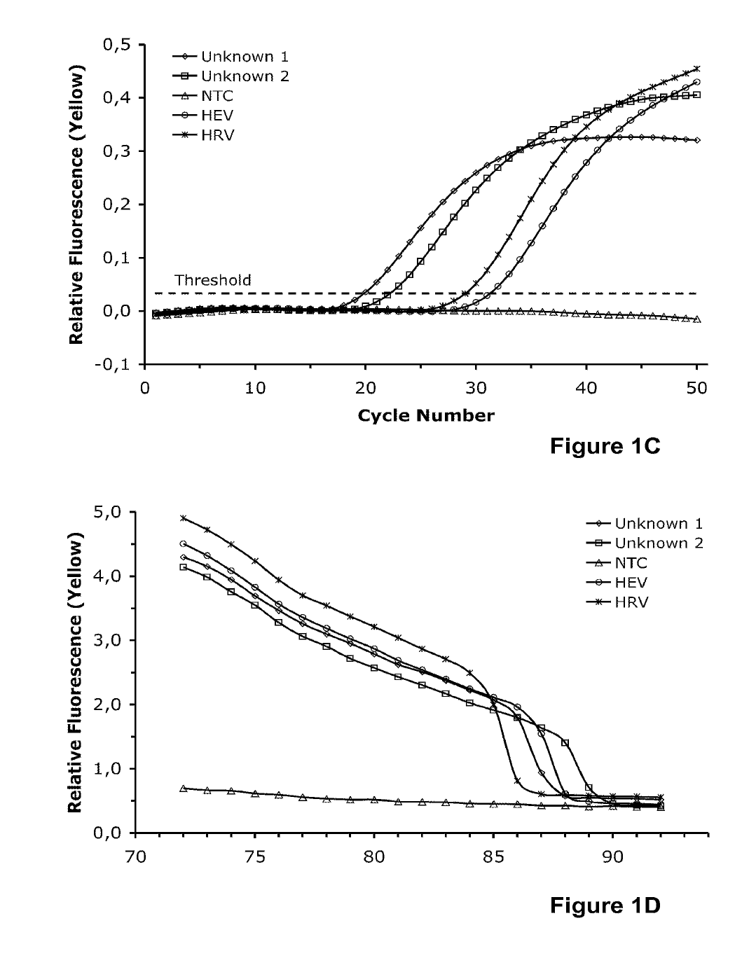

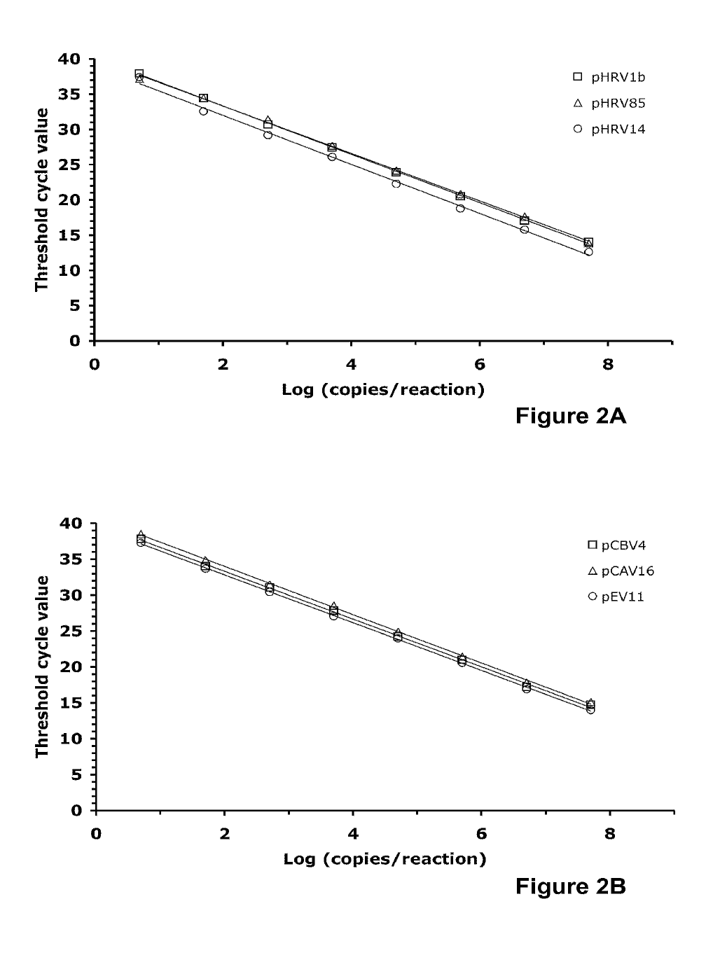

Results are illustrated in Tables 1A, 1B, 2A, 2B, 3 and 4, and FIGS. 1A to 1C, 2A and 2B.

### Table 1A and 1B

Prototype virus strains were cultured in appropriate cell types, until they showed a typical cytopathic effect throughout the cell layer. They were then subjected to three freeze-thaw cycles, and cell suspensions were clarified by centrifugation. The supernatants containing high titres of virus were stored at −70° C.

RNA from rhinovirus prototype strains was extracted from each supernatant as described in IIa above.

RNA was reverse transcribed to cDNA in an RT reaction as described in IIIa above.

DNA was amplified in PCR as described in IV above.

In each case of rhinovirus prototype strain tested, a Ct value indicative of a clearly positive reaction was observed in the green and yellow channels (Table 1A and 1B). In three cases, a Ct value higher than 40 was observed in the red channel, but in each of these three cases, the Ct value in the green channel was smaller by at least 16.

### Table 2A and 2B

Prototype virus strains were cultured in appropriate cell types, until they showed a typical cytopathic effect throughout the cell layer. They were then subjected to three freeze-thaw cycles, and cell suspensions were clarified by centrifugation. The supernatants containing high titres of virus were stored at −70° C.

RNA from enterovirus prototype strains was extracted from each supernatant as described in IIa above.

RNA was reverse transcribed to cDNA in an RT reaction as described in IIIa above.

DNA was amplified in PCR as described in IV above.

In each but one case of enterovirus prototype strain tested, a Ct value indicative of a clearly positive reaction was observed in the red and yellow channels (Table 2). One HEV-C strain, CAV1, gave a positive signal on yellow channel only, with a melting point typical to enteroviruses.

### Table 3

Clinical specimens were cultured in appropriate cell types, until they showed a typical cytopathic effect throughout the cell layer. Infected cell cultures were clarified by centrifugation and the supernatants with clinical isolates were stored at −70° C. Samples of specimens and clinical isolates suspected to contain enteroviruses were sent to the Enterovirus Laboratory, National Public Health Institute, Helsinki, Finland, for enterovirus typing. Specimens with an enterovirus type-specific result from the reference laboratory were selected to test the specificity of the new probe assay.

RNA from clinical isolates was extracted from each supernatant as described in IIb above.

RNA was reverse transcribed to cDNA in an RT reaction as described in IIIb above.

DNA was amplified in PCR as described in IV above.

In each but one case of the enterovirus isolates tested, a Ct value indicative of a clearly positive reaction was observed in the red and yellow channels (Table 3). In one case of CAV24, a relatively weak positive signal was obtained in the red channel only, indicating that in this case, the detection with probes was more sensitive than the detection with double-stranded DNA (dsDNA) dye. No false positive signals were obtained with rhinovirus specific probes in the green channel.

TABLE 1A
Results of testing rhinovirus prototype strains.

| | Prototype | Ct-HRV | Ct-HEV | Ct-dsDNA | Tm-dsDNA |
|---|---|---|---|---|---|
| Species | strain | (Green) | (Red) | (Yellow) | (Yellow) |
| HRV-A minor | 1A | 15.9 | | 18.5 | 85.50 |
| | 1B | 15.1 | | 17.3 | 85.70 |
| | 2 | 13.9 | | 15.1 | 85.70 |
| | 23 | 26.4 | | 24.5 | 86.50 |
| | 25 | 17.8 | | 19.0 | 85.20 |
| | 29 | 14.6 | | 15.2 | 86.30 |
| | 30 | 18.7 | | 17.6 | 86.50 |
| | 31 | 18.8 | | 15.7 | 85.15 |
| | 44 | 19.3 | | 20.1 | 86.80 |
| | 47 | 24.0 | | 23.5 | 86.00 |
| | 49 | 17.0 | | 15.8 | 86.50 |
| | 62 | 22.9 | | 22.6 | 85.00 |
| | 7 | 15.3 | | 17.8 | 86.20 |
| HRV-A major | 8 | 17.9 | | 20.3 | 85.80 |
| | 9 | 15.6 | | 17.0 | 86.20 |
| | 10 | 16.4 | | 13.7 | 87.80 |
| | 11 | 17.3 | 48.4 | 17.8 | 86.00 |
| | 12 | 16.0 | | 16.7 | 85.20 |
| | 13 | 28.3 | | 30.1 | 87.50 |
| | 15 | 19.0 | | 20.0 | 85.50 |
| | 16 | 19.7 | | 20.6 | 86.80 |
| | 18 | 20.9 | 43.1 | 22.0 | 86.50 |
| | 19 | 17.7 | | 19.7 | 86.80 |
| | 20 | 18.7 | | 19.9 | 85.50 |
| | 21 | 23.7 | | 20.9 | 86.00 |
| | Hanks-21 | 12.6 | | 12.8 | 87.50 |
| | 22 | 14.8 | | 16.5 | 86.30 |
| | 24 | 22.4 | | 23.0 | 85.80 |
| | 28 | 23.7 | | 25.5 | 84.50 |
| | 32 | 17.8 | | 19.3 | 86.50 |
| | 33 | 21.8 | | 22.5 | 85.15 |
| | 34 | 22.8 | | 24.0 | 87.00 |
| | 36 | 22.7 | | 24.4 | 88.00 |
| | 38 | 23.3 | | 24.5 | 85.80 |
| | 39 | 18.5 | | 20.4 | 86.00 |
| | 40 | 21.5 | | 22.3 | 85.70 |
| | 41 | 25.0 | | 24.4 | 86.32 |
| | 43 | 15.4 | | 17.0 | 85.70 |
| | 45 | 22.2 | | 22.3 | 86.70 |
| | 46 | 16.3 | | 14.4 | 86.20 |
| | 50 | 13.0 | | 13.2 | 86.00 |
| | 51 | 18.1 | | 17.2 | 86.00 |
| | 54 | 24.7 | | 24.6 | 86.00 |
| | 55 | 29.4 | | 28.2 | 86.30 |
| | 56 | 23.2 | | 23.0 | 86.50 |
| | 57 | 15.1 | | 14.6 | 87.50 |
| | 58 | 13.6 | | 16.6 | 88.00 |
| | 59 | 22.8 | | 24.3 | 85.30 |
| | 60 | 28.4 | | 27.1 | 85.20 |
| | 61 | 14.4 | | 13.8 | 87.80 |
| | 63 | 22.8 | | 23.1 | 87.30 |

TABLE 1B
Results of testing rhinovirus prototype strains.

| | Prototype | Ct-HRV | Ct-HEV | Ct-dsDNA | Tm-dsDNA |
|---|---|---|---|---|---|
| Species | strain | (Green) | (Red) | (Yellow) | (Yellow) |
| HRV-A major | 64 | 23.6 | | 23.6 | 86.30 |
| | 65 | 18.2 | | 17.8 | 86.50 |
| | 66 | 22.7 | | 22.3 | 86.00 |
| | 67 | 21.6 | | 19.1 | 87.50 |
| | 68 | 12.4 | | 13.7 | 87.00 |
| | 71 | 27.6 | | 25.9 | 85.00 |
| | 73 | 25.2 | | 26.1 | 85.80 |
| | 74 | 29.6 | | 29.3 | 86.00 |
| | 75 | 22.0 | | 23.3 | 86.00 |
| | 76 | 15.1 | | 14.8 | 85.70 |
| | 77 | 14.4 | | 14.7 | 85.30 |
| | 78 | 15.6 | | 14.8 | 85.30 |
| | 80 | 16.1 | | 15.8 | 85.80 |
| | 81 | 18.4 | | 14.8 | 87.00 |
| | 82 | 12.7 | | 14.2 | 86.50 |
| | 85 | 13.6 | | 13.6 | 86.50 |
| | 88 | 13.6 | | 14.7 | 86.80 |
| | 89 | 14.0 | | 16.4 | 87.30 |
| | 90 | 14.5 | | 15.4 | 86.80 |
| | 94 | 14.9 | | 17.1 | 86.30 |
| | 95 | 26.4 | | 28.1 | 85.50 |
| | 96 | 13.2 | | 16.5 | 87.00 |
| | 98 | 29.2 | | 29.7 | 85.80 |
| | 100 | 16.5 | | 17.0 | 87.00 |
| HRV-B | 3 | 17.3 | | 18.5 | 86.25 |
| | 4 | 17.2 | | 17.1 | 86.80 |
| | 5 | 15.1 | | 17.9 | 85.80 |
| | 6 | 20.1 | | 20.9 | 85.50 |
| | 17 | 14.7 | | 16.4 | 86.50 |
| | 26 | 17.8 | | 18.9 | 87.20 |
| | 27 | 13.7 | | 14.9 | 85.20 |
| | 35 | 27.6 | | 29.7 | 86.80 |
| | 37 | 13.4 | | 14.4 | 86.50 |
| | 42 | 27.5 | | 29.7 | 85.50 |
| | 48 | 30.1 | | 31.9 | 88.50 |
| | 52 | 26.8 | | 24.8 | 86.18 |
| | 57 | 32.9 | | 33.0 | 87.30 |
| | 69 | 31.1 | 47.4 | 31.5 | 88.20 |
| | 70 | 26.8 | | 27.9 | 87.00 |
| | 72 | 29.6 | | 32.2 | 86.50 |
| | 79 | 30.6 | | 32.2 | 86.00 |
| | 83 | 28.8 | | 30.4 | 86.30 |
| | 84 | 30.7 | | 32.3 | 86.00 |
| | 86 | 26.1 | | 30.2 | 86.00 |
| | 91 | 32.2 | | 32.7 | 85.80 |
| | 92 | 26.6 | | 27.7 | 86.70 |
| | 93 | 17.6 | | 19.5 | 85.50 |
| | 97 | 17.6 | | 20.5 | 86.20 |
| | 99 | 16.2 | | 17.7 | 87.20 |

TABLE 2A
Results of testing enterovirus prototype strains

| | Prototype | Ct-HRV | Ct-HEV | Ct-dsDNA | Tm-dsDNA |
|---|---|---|---|---|---|
| Species | strain | (Green) | (Red) | (Yellow) | (Yellow) |
| HEV-A | CAV 2 | | 23.4 | 20.8 | 87.8 |
| | CAV 3 | | 22.7 | 22.7 | 87.5 |
| | CAV 4 | | 20.7 | 20.8 | 88.0 |
| | CAV 5 | | 20.9 | 20.9 | 88.8 |
| | CAV 6 | | 24.5 | 24.4 | 88.0 |
| | CAV 7 | | 22.3 | 24.3 | 90.7 |
| | CAV 8 | | 18.4 | 18.5 | 88.0 |
| | CAV 10 | | 23.5 | 26.7 | 89.0 |
| | CAV 12 | | 21.9 | 22.1 | 86.5 |
| | CAV 14 | | 29.4 | 28.1 | 86.8 |
| | CAV 16 | | 20.3 | 21.4 | 89.7 |
| | EV 71 | | 19.9 | 21.2 | 86.1 |
| HEV-B | CBV 1 | | 23.6 | 22.6 | 88.5 |
| | CBV 2 | | 31.2 | 29.3 | 88.5 |
| | CBV 3 | | 21.7 | 21.4 | 89.3 |
| | CBV 4 | | 23.1 | 23.5 | 89.5 |
| | CBV 5 | | 21.0 | 21.5 | 89.7 |
| | CBV 6 | | 36.4 | 36.1 | 88.8 |
| | CAV 9 | | 22.1 | 22.6 | 90.5 |
| | ECHO 1 | | 32.9 | 27.9 | 87.0 |
| | ECHO 2 | | 13.0 | 12.7 | 88.3 |
| | ECHO 3 | | 27.3 | 26.5 | 86.5 |
| | ECHO 4 | | 28.3 | 28.4 | 87.5 |
| | ECHO 5 | | 20.2 | 19.8 | 88.2 |
| | ECHO 6 | | 25.0 | 26.8 | 87.5 |
| | ECHO 7 | | 22.1 | 24.4 | 86.5 |
| | ECHO 8 | | 16.7 | 17.1 | 88.3 |
| | ECHO 9 | | 22.1 | 25.6 | 87.5 |
| | ECHO 11 | | 24.6 | 26.8 | 88.2 |
| | ECHO 12 | | 19.4 | 18.9 | 87.5 |
| | ECHO 13 | | 16.0 | 16.2 | 87.2 |
| | ECHO 14 | | 19.0 | 19.7 | 88.2 |
| | ECHO 15 | | 16.0 | 16.5 | 87.5 |
| | ECHO 16 | | 15.3 | 14.6 | 88.5 |
| | ECHO 17 | | 15.1 | 17.1 | 88.8 |
| | ECHO 18 | | 28.2 | 21.5 | 87.7 |
| | ECHO 19 | | 11.9 | 12.2 | 87.8 |
| | ECHO 20 | | 12.9 | 14.5 | 88.5 |
| | ECHO 21 | | 13.5 | 13.2 | 88.5 |
| | ECHO 24 | | 28.3 | 23.0 | 88.5 |
| | ECHO 25 | | 23.0 | 21.7 | 88.5 |
| | ECHO 26 | | 24.6 | 23.9 | 88.8 |
| | ECHO 27 | | 17.0 | 17.0 | 88.0 |
| | ECHO 29 | | 22.4 | 22.2 | 87.0 |
| | ECHO 30 | | 21.9 | 21.3 | 88.8 |
| | ECHO 32 | | 27.9 | 27.5 | 86.7 |
| | ECHO 33 | | 13.6 | 14.5 | 87.8 |

TABLE 2B
Results of testing enterovirus prototype strains

| | Prototype | Ct-HRV | Ct-HEV | Ct-dsDNA | Tm-dsDNA |
|---|---|---|---|---|---|
| Species | strain | (Green) | (Red) | (Yellow) | (Yellow) |
| HEV-C | CAV 1 | | — | 34.0 | 88.2 |
| | CAV 11 | | 38.0 | 28.8 | 87.0 |
| | CAV 13 | | 25.9 | 22.0 | 86.8 |
| | CAV 15 | | 26.2 | 21.7 | 88.5 |
| | CAV 17 | | 18.0 | 15.2 | 87.5 |
| | CAV 18 | | 17.6 | 14.8 | 86.7 |
| | CAV19 | | 17.6 | 15.6 | 87.7 |
| | CAV20A | | 31.8 | 28.6 | 86.2 |
| | CAV 20B | | 26.7 | 23.7 | 87.0 |
| | CAV 21 | | 25.4 | 22.1 | 88.2 |
| | CAV 22 | | 33.3 | 29.7 | 86.5 |
| | CAV 24 | | 23.0 | 19.7 | 87.3 |
| HEV-D | EV 68 | | 15.0 | 10.8 | 88.2 |
| Polioviruses* | PV 1 | | 23.4 | 17.1 | 90.2 |
| | PV 2 | | 28.1 | 24.5 | 89.5 |
| | PV 3 | | 26.1 | 18.1 | 90.5 |

*Vaccine strains

### Table 4

To demonstrate the assay specificity in detection and identification of rhino- and enteroviruses directly in clinical specimens, picornavirus positive specimens were subjected to nucleic acid sequence analysis as described in (Peltola et. al., Infect Dis. 2008; 1; 197:382-9) and (Nix et al., J Clin Microbiol. 2006; 44:2698-704).

According to sequence analysis, picornaviruses in the specimens were typed as rhinovirus A, B, or C, or enterovirus (nearest prototype strain).

RNA from each clinical specimen was extracted as described in IIb. RNA was reverse transcribed to cDNA in an RT reaction as described in IIIb.

DNA was amplified in PCR as described in IV.

In each case, the result of PCR with the new probes was concordant with the sequence based typing result. Of particular interest is the fact that the method detects group C rhinoviruses, which are not cultivatable in cell culture, and thus, no prototype strains are assigned or available for them.

TABLE 3
Results of testing cell cultures inoculated with clinical specimens.
Reference results were obtained from the Enterovirus Laboratory, NPHI, Helsinki, Finland.

| | Type of the original | Ct-HRV | Ct-HEV | Ct-dsDNA | Tm-dsDNA | Reference result |
|---|---|---|---|---|---|---|
| No. | specimen | (Green) | (Red) | (Yellow) | (Yellow) | Virus (type) |
| 1 | Biopsy | | 14.0 | 12.3 | 87.2 | HEV (EV71) |
| 2 | Cereprospinal fluid | | 12.5 | 10.8 | 87.5 | HEV (E30) |
| 3 | Conjuctival fluid | | 35.5 | | | HEV (CAV24) |
| 4 | Stool | | 20.8 | 15.7 | 88.0 | HEV (CAV24) |
| 5 | Stool | | 13.9 | 12.1 | 88.5 | HEV (E2) |
| 6 | Stool | | 15.2 | 13.3 | 89.5 | HEV (CBV4) |
| 7 | Stool | | 12.2 | 13.2 | 89.5 | HEV (CBV4) |
| 8 | Stool | | 20.1 | 13.1 | 88.7 | HEV (CAV24) |
| 9 | Stool | | 19.7 | 17.1 | 87.5 | HEV (EV71) |
| 10 | Stool | | 18.6 | 12.0 | 89.0 | HEV (PV2&PV3)* |
| 11 | Stool | | 16.0 | 13.8 | 86.7 | HEV (E9) |
| 12 | Stool | | 36.9 | 29.8 | 86.4 | HEV (CAV24) |
| 13 | Stool | | 15.7 | 8.5 | 84.3 | HEV (CAV9&CAV24) |
| 14 | Stool | | 15.7 | 11.3 | 85.8 | HEV (CAV24) |
| 15 | Stool | | 30.1 | 29.2 | 87.0 | HEV (E20) |
| 16 | Stool | | 32.7 | 28.6 | 86.2 | HEV (CAV24) |

*Sabin (oral poliovirus vaccine) strains.

TABLE 4
Results of direct testing of clinical specimens.
Reference result obtained by partial sequencing of the amplified virus genome.

| | | Ct-HRV | Ct-HEV | Ct-dsDNA | Tm-dsDNA | Reference result |
|---|---|---|---|---|---|---|
| No. | Type of the specimen | (Green) | (Red) | (Yellow) | (Yellow) | Virus (type) |
| 1 | Bronchoalveolar lavage | 33.2 | | 28.9 | 84.7 | HRV-A |
| 2 | Cereprospinal fluid | | 33.4 | 31.8 | 86.0 | HEV (EV71) |
| 3 | Nasal lavage | 26.5 | | 27.1 | 85.5 | HRV-C |
| 4 | Nasal swab | 29.8 | | 26.9 | 87.2 | HRV-B |
| 5 | Nasal swab | 28.9 | | 29.6 | 87.2 | HRV-A |
| 6 | Nasal swab | | 22.4 | 22.3 | 87.5 | HEV (CAV16) |
| 7 | Nasopharyngeal aspirate | 15.6 | | 15.7 | 85.2 | HRV-C |
| 8 | Nasopharyngeal aspirate | 22.2 | | 22.2 | 86.2 | HRV-C |
| 9 | Nasopharyngeal aspirate | 23.0 | | 19.3 | 85.7 | HRV-A |
| 10 | Stool | | 26.5 | 24.6 | 87.2 | HEV (CAV16) |
| 11 | Throat swab | 25.7 | | 21.9 | 87.7 | HRV-B |
| 12 | Throat swab | 32.2 | | 26.5 | 84.0 | HRV-A |
| 13 | Throat swab | | 32.0 | 30.4 | 87.0 | HEV (CAV16) |
| 14 | Throat swab | | 21.1 | 22.6 | 87.2 | HEV (CAV6) |
| 15 | Vesicular fluid | | 22.9 | 22.56 | 87.2 | HEV (CAV6) |
| 16 | Vesicular fluid | | 41.8 | 39.8 | 85.0 | HEV (untyped) |

### FIG. 1

The principle of a RT-PCR assay is illustrated in FIGS. 1A to 1D.

Positive controls for rhinovirus (HRV) and enterovirus (HEV) RNA were prepared using prototype strains HRV1b and E11 as described for Table 1A and 1B, and stored at −70° C. RNA from clinical specimens (unknown 1 and 2) and no template control (NTC), containing water instead of specimen were extracted as described in IIb above.

RNA was reverse transcribed to cDNA in an RT reaction as described in IIIb above.

DNA was amplified in PCR as described in IV above.

In FIG. 1A, the fluorescence of FAM was measured on the green channel, and fluorescence increase and amplification curves were obtained for HRV positive control (crosses) and unknown 1 (diamonds). A threshold could be set to clearly separate the background fluorescence from that of the true amplifications. In this case, the clinical sample unknown 1 was shown to contain rhinovirus.

In FIG. 1B, the fluorescence of Cy5 was measured on the red channel, and fluorescence increase and amplification curves were obtained for HEV positive control (circles) and unknown 2 (squares). A threshold could be set to clearly separate the background fluorescence from that of the true amplifications. In this case, the clinical sample unknown 2 was shown to contain enterovirus.

In FIG. 1C, the fluorescence of dsDNA dye BOXTO was measured on the yellow channel, and fluorescence increase and amplification curves were obtained for positive controls and unknowns. A threshold could be set to clearly separate the background fluorescence from that of the true amplifications.

FIG. 1D shows the melting curves measured on the yellow channel corresponding decrease of binding of BOXTO to dsDNA, as temperature increases. A sharp drop of the fluorescence is observed at the melting point of the DNA amplicons. In this case, unknown 1 (diamonds) has a relatively low melting point typical to rhinoviruses, and unknown 2 (squares) has a relatively high melting point typical to enteroviruses.

### FIG. 2

Plasmids pHRV1b, pHRV14, and pHRV85, containing cDNA copies of corresponding rhinoviruses (FIG. 2A) or plasmids pCBV4, pCAV16, and pEV11, containing corresponding enteroviruses (FIG. 2B) were used in the PCR as described in 1V. A ten-fold dilution series was prepared for each type of plasmid, so that PCR reactions contained 5 to 50 000 000 plasmid copies corresponding as many virus genome copies per reaction. Threshold cycle values were recorded in green channel for rhinovirus genome containing plasmids and in red channel for enterovirus genome containing plasmids. Values of log(copies/reaction) and threshold cycle for each dilution was plotted in an xy-graph and linear regression line was calculated for each series. From the slope of the linear regression line, the PCR efficiency (E) can be calculated by solving from the formula:

E=10^{(-1/slope)}−1, (1)

where

Reaction efficiency indicates the average fraction of duplicated copies during each cycle. Thus, in a perfect PCR, E=1, also given as 100%.

Efficiencies for rhinovirus cDNA amplification calculated from the standard curves in FIG. 2B were 96%, 94%, and 98%, for pHRV1b, pHRV14, and pHRV85, respectively.

Efficiencies for enterovirus cDNA amplification calculated from the standard curves in FIG. 2B were 100%, 98%, and 100%, for pCBV4, pCAV16, and pEV11, respectively.

These results indicate that the described probes can be used in a quantitative assay with a wide dynamic range.

### Other Preferred Embodiments

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. A nucleic acid comprising a 13 base sequence selected from the group consisting of a) tcGgTtccgCtGc (SEQ ID NO: 1), b) tcGgTtccgCcAc (SEQ ID NO: 2), c) tcGgTcCcaTcCc (SEQ ID NO: 3), d) tcGgTtCcaTcCc (SEQ ID NO: 4), e) ttGgTcCcaTcCc (SEQ ID NO: 5), f) ttGgTtCcaTcCc (SEQ ID NO: 6), g) tcGgTcccgTcCc (SEQ ID NO: 7), h) tcGgTtccgTcCc (SEQ ID NO: 8), wherein A and a are adenine, C and c are cytosine, G and g are guanine, and T and t are thymine or uracil, and provided that in any case the nucleic acid does not comprise more than 10 additional bases at either the 5′ end or the 3′ end of the nucleic acid; and ii) if the nucleic acid comprises alternative a) said nucleic acid does not comprise any additional bases at the 5′ end; or, preferably and, does not comprise more than 4, preferably not more than 3, more preferably not more than 2, even more preferably not more than 1 and most preferable not any additional bases at the 3′ end; ii) if the nucleic acid comprises alternative b) said nucleic acid does not comprise more than 3, preferably not more than 2, more preferably not more than 1 and most preferable not any additional bases at the 5′ end; or, preferably and, does not comprise more than 5, preferably not more than 3, more preferably not more than 2, even more preferably not more than 1 and most preferable not any additional bases at the 3′ end; iii) if the nucleic acid comprises alternative c) said nucleic acid does not comprise (1) any, (2) more than 2, or (3) more than 4 additional bases at the 5′ end, and does not comprise (1) more than 6, (2) more than 4, or (3) more than 2, respectively, additional bases at the 3′ end, and preferably does not comprise any additional bases at either said 5′end or said 3′ end; iv) if the nucleic acid comprises alternative d) said nucleic acid does not comprise more than 2, more preferably not more than 1 and most preferable not any additional bases at the 5′ end; or, preferably and, does not comprise more than 2, preferably not more than 1 and most preferable not any additional bases at the 3′ end; v) if the nucleic acid comprises alternative e) said nucleic acid does not comprise any additional bases at the 5′ end; or, preferably and, not any additional bases at the 3′ end; vi) if the nucleic acid comprises alternative f) said nucleic acid does not comprise more than 3, preferably not more than 2, more preferably not more than 1 and most preferable not any additional bases at the 5′ end; or, preferably and, does not comprise more than 10, preferably not more than 3, more preferably not more than 2, even more preferably not more than 1 and most preferable not any additional bases at the 3′ end; vii) if the nucleic acid comprises alternative g) said nucleic acid does not comprise more than 10, preferably not more than 3, more preferably not more than 2, even more preferably not more than 1 and most preferable not any additional bases at the 5′ end; and does not comprise any additional bases at the 3′ end; and viii) if the nucleic acid comprises alternative h) said nucleic acid does not comprise more than 10, preferably not more than 3, more preferably not more than 2, even more preferably not more than 1 and most preferable not any additional bases at the 5′ end; or, preferably and, does not comprise any additional bases at the 3′ end; and complementary sequences thereof.

2. The nucleic acid according to claim 1 wherein said nucleic acid comprises modified nucleotides, preferably locked nucleotides.

3. The nucleic acid according to claim 2 wherein the nucleic acid comprises locked nucleotides, preferably at positions with the capital letters A, C, G, T denoting the base of said locked nucleotides.

4. The nucleic acid according to claim 3 wherein the nucleic acid comprises 2 to 7, preferably 3 to 6, most preferably 4 or 5 locked nucleotides.

5. The nucleic acid according to claim 4 wherein at least nucleotides 10 and 12 of sequences a) to h) and at least nucleotides 2 and 4 of sequences complementary to said sequences a) to h) are locked.

6. The nucleic acid according to claim 1, wherein said nucleic acid is a DNA, a RNA, a LNA, or PNA preferably a LNA or PNA, and most preferably a LNA.

7. Use of nucleic acids comprising a 13 base sequence selected from the group consisting of a) tcGgTtccgCtGc (SEQ ID NO: 1), b) tcGgTtccgCcAc (SEQ ID NO: 2), c) tcGgTcCcaTcCc (SEQ ID NO: 3), d) tcGgTtCcaTcCc (SEQ ID NO: 4), e) ttGgTcCcaTcCc (SEQ ID NO: 5), f) ttGgTtCcaTcCc (SEQ ID NO: 6), g) tcGgTcccgTcCc (SEQ ID NO: 7), h) tcGgTtccgTcCc (SEQ ID NO: 8), wherein A and a are adenine, C and c are cytosine, G and g are guanine, and T and t are thymine or uracil; and provided that in any case the nucleic acid does not comprise more than 30, preferably not more than 10, more preferably not more than 3, even more preferably not more than 1 and most preferable not any additional bases at the 5′ end; and does not comprise more than 30, preferably not more than 10, more preferably not more than 3, even more preferably not more than 1 and most preferable not any additional bases at the 3′ end; and complementary sequences thereof; for differentiating picornaviruses between enteroviruses and rhinoviruses, wherein at least 2, preferably at least 4, more preferably at least and most preferably 8 different said nucleic acids, each corresponding to one of said nucleic acids with one of sequences a) to h) or said complementary sequence thereof provided that only one of each pair of said sequence and its complementary sequence is used.

8. The use according to claim 7 wherein the nucleic acids used are nucleic acids as defined by claim 1.

9. The use according to claim 7 wherein the nucleic acids used do not comprise any additional nucleotides to those defined by the 13 base sequences defined by alternatives a) to h) and the complementary sequences thereof.

10. The use according to claim 7, wherein one or more of the nucleic acids used comprise modified nucleotides, preferably locked nucleotides.

11. The use according to claim 10 wherein one or more of the nucleic acids comprises locked nucleotides, preferably at positions designated with the capital letters A, C, G, T denoting the base of said locked nucleotides.

12. The use according to claim 11 wherein one or more of the nucleic acids used comprise 2 to 7, preferably 3 to 6, most preferably 4 or 5 locked nucleotides.

13. The use according to claim 12 at least nucleotides 10 and 12 of sequences a) to h) and at least nucleotides 2 and 4 of sequences complementary to said sequences a) to h) are locked.

14. The use according to claim 7, wherein at least one of the nucleic acids used is a DNA, a RNA, a LNA, or a PNA preferably a LNA or a PNA, and most preferably a LNA.

15. The use according to claim 7, wherein a picornavirus, which cannot be recognized by the probes used, is detected employing a general dsDNA label; and, by determining the melting point, differentiated, based on said melting point, to be either an enterovirus or rhinovirus.

16. Use of any of the nucleic acids defined in claim 1 as a determiner portion in a priming oligonucleotide for differentiating picornaviruses, preferably between enteroviruses and rhinoviruses.

17. A bioassay method wherein picornaviruses are differentiated between enteroviruses and rhinovirus wherein the nucleic acids defined by claim 1 are employed in a nucleic acid amplification assay.

18. The bioassay method of claim 17 wherein the nucleic acid amplification assay is a polymer chain reaction (PCR) assay or a nucleic acid sequence based amplification assay (NASBA), preferably a PCR assay.

19. The bioassay method of claim 18 wherein the nucleic acid amplification assay is a PCR assay which comprises the steps of a) nucleic acid extraction from a sample, b) reverse transcription, c) PCR amplification employing nucleic acids defined by claim 1, and d) analysis of amplification results.

20. The bioassay method of claim 18 at least one nucleic acid as defined in a) and b) and at least one nucleic acid as defined in c) to g) are employed, and ii) said nucleic acid as defined in a) and b) are labelled with a first label detectable at a first wavelength and said nucleic acids as defined in c) to g) are labelled with a second label detectable at a second wavelength.

21. The bioassay method of claim 20 wherein a double stranded nucleic acid dye detecting any nucleic acid amplification, preferably at a third wavelength, is further employed.

22. The bioassay method of claim 19, wherein in step c) a multichannel real time PCR instrument employing at least two, preferably at least three, different wavelength channels is used.

23. The bioassay method of claim 19, wherein step d) comprises melting point determination of the end products of PCR amplification.

24. The bioassay method of claim 20, wherein dilution series of samples are employed and results are compared to standards with known copy numbers in order to obtain quantitative results.


SEQUENCE LISTING (10 sequences)

SEQ ID NO: 1 - DNA, 13 bp, Artificial
  Probe a
tcggttccgctgc

SEQ ID NO: 2 - DNA, 13 bp, Artificial
  Probe b
tcggttccgccac

SEQ ID NO: 3 - DNA, 13 bp, Artificial
  Probe c
tcggtcccatccc

SEQ ID NO: 4 - DNA, 13 bp, Artificial
  Probe d
tcggttccatccc

SEQ ID NO: 5 - DNA, 13 bp, Artificial
  Probe e
ttggtcccatccc

SEQ ID NO: 6 - DNA, 13 bp, Artificial
  Probe f
ttggttccatccc

SEQ ID NO: 7 - DNA, 13 bp, Artificial
  Probe g
tcggtcccgtccc

SEQ ID NO: 8 - DNA, 13 bp, Artificial
  Probe h
tcggttccgtccc

SEQ ID NO: 9 - DNA, 21 bp, Artificial
  ENRI(4-) primer
gaaacacggacacccaaagta

SEQ ID NO: 10 - DNA, 19 bp, Artificial
  ENRI(3+) primer
cggccctgaatgcggctaa